(19) United States
(12) Patent Application Publication
(10) Pub. No.: US 2024/0054725 A1
(43) Pub. Date: Feb. 15, 2024

(54) Method of siting emergency medical service facilities in mountain villages

(71) Applicant: Henan University, Kaifeng City (CN)

(72) Inventors: Yulong CHEN, Kaifeng City (CN); Zhizhu LAI, Kaifeng City (CN)

(21) Appl. No.: 18/446,525

(22) Filed: Aug. 9, 2023

(30) Foreign Application Priority Data
Aug. 9, 2022 (CN) 202210947405.0

(51) Int. Cl. G06T 17/05; G16H 40/20

(52) U.S. Cl. CPC G06T 17/05; G16H 40/20

(57) ABSTRACT

A method of siting emergency medical service facilities in mountain villages, including: obtaining mountainous village spatial data information; determining a location corresponding to each of a plurality of alternative facility sites in the predetermined area based on the mountainous village spatial data information; determining a mountainous emergency care efficiency set corresponding to each settlement in the predetermined area based on the mountainous village spatial data information; constructing a mountainous village emergency care model; and determining road optimization information and a location and a number of newly constructed facility sites based on the mountainous village spatial data information and the mountainous village emergency care model.

### TECHNICAL FIELD

The disclosure relates to the field of geographic information technology, and specifically relates to a method for selecting the location of emergency care service facility in mountainous village.

### BACKGROUND ART

China's mountainous areas account for 69.1% of the country's total land area, and a considerable portion of the countryside is established in mountainous areas, which have complex topography, are usually prone to geological disasters such as collapses, landslides and mudslides, and have lagging road networks and high vulnerability, and are also weak areas in terms of medical resources. For a long time, there is a huge gap between urban and rural medical service facilities in China, and there is a shortage of medical resources in rural areas, and there is a structural imbalance between supply and demand, which seriously restricts the coordination between urban and rural areas and the sustainable development of rural areas. Emergency care service facilities are an important part of China's health service system, and there is a shortage of first-aid stations in rural areas and the problem that first-aid stations are often concentrated in cities. Meanwhile, transportation in mountainous areas is susceptible to natural disasters, complex mountainous environments and weather conditions, which leads to the vulnerability and variability of mountain road networks and increases the difficulty of medical emergencies. In the face of sudden-onset natural disasters or public health incidents, some remote rural areas located in mountainous environments, due to the limitations of transportation conditions, patients or the casualty often cannot get medical assistance in the first time, and miss the best rescue time of 15 minutes for medical assistance (critical time for emergency care).

The planning and layout of emergency care service facilities in rural areas often lacks systematic, effective and targeted guidance that is responsive to changes in development, the irrational layout of emergency care service facilities may often result in low utilization of facilities, and facilities are too large or too small in scale, leading to a waste of resources and increased costs. Therefore, the scientific layout of emergency care facilities in mountainous villages and convenient transportation networks play a crucial role in shortening the time and improving the efficiency of medical emergencies, which is directly related to the health and life safety of the majority of rural settlements.

At present, most of the siting problems of emergency care service facilities are based on the classical maximum coverage model for spatial location siting simulation, often ignoring the optimization design of the transportation network, which cannot meet the special needs of emergency care service facilities located in mountainous environments. For the spatial configuration and optimization of emergency care facilities in mountain villages, it is necessary to explore an optimization method that can be highly integrated with the characteristics of mountain villages. For the layout of medical emergency service facilities in mountain villages, the present solution takes into account the complexity of road conditions, the fragility and variability of the road network, and the accessibility of the transportation network.

Therefore, the disclosure combines the facility location and network design method, introduces the multi-objective optimization theory based on the characteristics of mountain villages themselves, constructs the siting model of emergency care service facilities in mountain villages in a targeted manner, and applies it to solve the problem of optimizing the layout of emergency care service facilities in mountain villages, so as to improve the reasonableness of the optimized layout of emergency care service facilities in mountain villages.

### DISCLOSURE

The contents of the disclosure are used to introduce in brief form ideas which will be described in detail later in the specific embodiments. The content of the disclosure is not intended to identify the key features or necessary features of the technical solution for which protection is claimed, nor is it intended to be used to limit the scope of the technical solution for which protection is claimed.

In order to solve the technical problem of low rationality in determining the number and location of emergency care service facilities, the disclosure proposes a method for selecting location of emergency care service facilities in mountain villages.

The disclosure provides a method of siting emergency medical service facilities in mountain villages, comprising:
obtaining mountainous village spatial data information, wherein the mountainous village spatial data information is information corresponding to a geographic situation within a predetermined area, and the mountainous village spatial data information comprises: settlement information corresponding to each settlement in the predetermined area, emergency care facility site information corresponding to an emergency care facility site, and road information corresponding to a road;
determining a location corresponding to each of a plurality of alternative facility sites in the predetermined area based on the mountainous village spatial data information;
determining a mountainous emergency care efficiency set corresponding to each settlement in the predetermined area based on the mountainous village spatial data information;
constructing a mountainous village emergency care model based on locations corresponding to the plurality of alternative facility sites and the mountainous emergency care efficiency set corresponding to a settlement in the predetermined area; and
determining road optimization information and location and number of newly constructed facility sites based on the mountainous village spatial data information and the mountainous village emergency care model.

Further, the obtaining mountainous village spatial data information, comprising:
obtaining historical spatial data information, wherein the historical spatial data information is recorded and corresponds to a geographic situation in the predetermined area under a closest moment of a current moment;
obtaining, by means of a remote sensing device, a remote sensing image in the predetermined area under the current moment; and
updating the historical spatial data information based on the remote sensing image to obtain the mountainous village spatial data information.

Further, the determining the location corresponding to each of the plurality of alternative facility sites in the predetermined area based on the mountainous village spatial data information, comprising:
dividing the predetermined area into a plurality of evaluation units;
determining a plurality of influencing factors and weights corresponding to the plurality of influencing factors;
determining a plurality of indicators corresponding to each evaluation unit of the plurality of evaluation units based on the mountainous village spatial data information and the plurality of influencing factors;
determining an indicator impact score corresponding to each indicator of the plurality of indicators corresponding to each evaluation unit of the plurality of evaluation units based on the plurality of indicators corresponding to each evaluation unit of the plurality of evaluation units;
determining a total indicator impact score corresponding to each of the plurality of evaluation units based on the weights corresponding to the plurality of influencing factors and the indicator impact score corresponding to each indicator of the plurality of indicators corresponding to each of the evaluation units in the plurality of evaluation units; and
determining a location corresponding to each of the plurality of alternative facility sites in the predetermined area based on the total indicator impact score corresponding to the plurality of evaluation units.

Further, a formula for determining the total indicator impact score corresponding to each of the plurality of evaluation units corresponds to:

$${P}_{φ} = {\sum }_{σ = 1}^{n} {F}_{φσ} \times {w}_{σ}$$

where P_{φ} is the total indicator impact score corresponding to φₜₕ evaluation unit, n is a number of influencing factors among the plurality of influencing factors, F_{φσ} is σₜₕ indicator corresponding to the φₜₕ evaluation unit, and w_{σ} is a weight corresponding to σₜₕ influencing factor.

Further, the determining the mountainous emergency care efficiency set corresponding to each settlement in the predetermined area based on the mountainous village spatial data information, comprising:
determining a time satisfaction of transferring a patient or an casualty in a settlement to each facility site to obtain a time satisfaction set corresponding to the settlement based on the mountainous village spatial data information, wherein the facility site is an alternative facility site or an emergency care facility site;
determining road vulnerability coefficients of road sections through which the patient or casualty in the settlement is transferred to each facility site to obtain a road vulnerability coefficient set corresponding to the settlement based on the information of hidden roads included in the mountainous village spatial data information;
determining traffic satisfaction of the road sections through which the patient or casualty in the settlement are transferred to each facility site to obtain a traffic satisfaction set corresponding to the settlement based on traffic condition information included in the mountainous village spatial data information; and
determining a mountainous emergency care efficiency set corresponding to the settlement based on the time satisfaction set, the road vulnerability coefficient set and the traffic satisfaction set corresponding to the settlement.

Further, the determining the time satisfaction for transferring a patient or casualty in the settlement to each facility site based on the mountainous village spatial data information, comprising:
determining an access road set from the settlement to each facility site based on the mountainous village spatial data information;
determining a formular corresponding to a first time satisfaction set between the settlement and the facility site based on the access road set from the settlement to each facility site and a pre-obtained time spent cost of each road in the access road set as:

$$\text{?} = \text{?} {tr}_{ij}$$

$$\text{?} \text{indicates text missing or illegible when filed}$$

where G_{k,a,A} is the first time satisfaction of the patient or casualty in a kₜₕ settlement transferred to an Aₜₕ facility site in the predetermined area through an aₜₕ access road, the aₜₕ access road is an access road from the kₜₕ settlement to the Aₜₕ facility in the predetermined area, L_{k,a,A} is all the roads which make up the aₜₕ access road from the kₜₕ settlement to the Aₜₕ facility site in the predetermined area, trᵢⱼ is a time cost for the patient or casualty to pass through a road with road link (i,j) in L_{k,a,A,} and the first time satisfaction set between the kₜₕ settlement and the Aₜₕ facility site in the predetermined area includes: the first time satisfaction of the patient or casualty in the kₜₕ settlement who is transferred to the Aₜₕ facility site through each access road;
determining a smallest first time satisfaction in the first time satisfactions set from the settlement to each facility site as a reference time satisfaction from the settlement to the facility site;
combining reference time satisfactions from the settlement to each facility site into a reference time satisfaction set corresponding to the settlement;
determining a largest reference time satisfaction in the reference time satisfaction set corresponding to the settlement as a control time satisfaction corresponding to the settlement; and
determining a formula corresponding to the time satisfaction for a transfer of the patient or casualty from the settlement to each facility site based on the control time satisfaction corresponding to the settlement and the reference time satisfaction set from the settlement to each facility site as:

$${g}_{k , A} = \frac{{G}_{k} - {G}_{k , A}}{{G}_{k}}$$

where g_{k,A} is a time satisfaction for the transfer of the patient or casualty in the kₜₕ settlement to the Aₜₕ facility site in the predetermined area, Gₖ is a control time satisfaction corresponding to the kₜₕ settlement in the predetermined area, and G_{k,A} is a reference time satisfaction from the kₜₕ settlement to the Aₜₕ facility site in the predetermined area.

Further, a formula for determining the road vulnerability coefficient corresponding to the road section through which the patient or casualty in the settlement is transferred to each facility site is:

$${ρ}_{k , A} = \frac{{L}_{k , A}}{l}$$

where ρ_{k,A} is a road vulnerability coefficient of the road section through which the patient or casualty from the kₜₕ settlement is transferred to the Aₜₕ facility site in the predetermined area, L_{k,A} is a number of potentially hazardous roads among all the roads from the kₜₕ settlement to Aₜₕ facility site in the predetermined area, and l is a number of potentially hazardous roads among all the roads in the predetermined area.

Further, a formula for determining the mountainous emergency care efficiency set corresponding to the settlement is:

$${F}_{k , A} = \{ \begin{matrix}1 & {G}_{k , A} = 0 \\ ( 1 - {ρ}_{k , A} ) ( α {g}_{k , A} + β {λ}_{k , A} ) & {G}_{k , A} > 0\end{matrix}$$

where F_{k,A} is an Aₜₕ mountainous emergency care efficiency in the mountainous emergency care efficiency set corresponding to the kₜₕ settlement in the predetermined area, α and β are weight coefficients, α∈[0,1], β∈[0,1], α+β=1, G_{k,A} is a reference time satisfaction from the kₜₕ settlement to the Aₜₕ facility site in the predetermined area, ρ_{k,A} is a road vulnerability coefficient of the road through which the patient or casualty of the kₜₕ settlement is transferred to the Aₜₕ facility site in the predetermined area, g_{k,A} is a time satisfaction of the patient or casualty in the kₜₕ settlement transferred to the Aₜₕ facility site in the predetermined area, and λ_{k,A} is a traffic satisfaction of the road section through which the patient or casualty in the kₜₕ settlement is transferred to the Aₜₕ facility site in the predetermined area.

Further, the determining the road optimization information and the location and number of newly constructed facility sites based on the mountainous village spatial data information and the mountainous village emergency care model comprises:
determining the road optimization information and the location and number of newly constructed facility sites based on the mountainous village spatial data information and the mountainous village emergency care model, by means of an improved multi-objective simulated annealing algorithm.

The disclosure has the following advantageous effects:
the method for selecting the location of emergency care service facility in mountainous village of the disclosure improves the rationality of the location of a road section in need of transportation improvement and the location and number of emergency care service facilities, and improves the coverage rate and emergency care service efficiency of the emergency care service under the input of limited cost in the mountainous village. First, a spatial data information of the mountainous village is obtained, wherein the afore spatial data information of the mountainous village is information corresponding to a geographic situation within a predetermined area, and the afore spatial data information of the mountainous village comprises: information of a settlement corresponding to each settlement within the afore predetermined area, information of an emergency care facility site corresponding to the emergency care facility site, and information of a road corresponding to the road. In fact, for selecting the location of the emergency care facility site, it is often necessary to obtain information about the area in which the emergency care facility site needs to be added, which can facilitate comparison of the geographic conditions of different areas in the predetermined area, and can improve the accuracy of the subsequent selection of the location of the emergency care facility site. Next, based on the afore spatial data information of the mountainous village, a location corresponding to a plurality of alternative facility sites within the above predetermined area is determined. A plurality of factors are considered in a comprehensive manner, which improves the accuracy of the preliminary determination of the locations corresponding to the alternative facility sites. Then, based on the above mountainous village spatial data information, a mountainous emergency care efficiency set corresponding to each settlement within the above predetermined area is determined. Afterwards, mountainous villages emergency care model is constructed based on the locations corresponding to the plurality of alternative facility sites and the mountainous emergency care efficiency set corresponding to the settlements within the above-described predetermined area. Finally, based on the above mountainous village spatial data information and the above mountainous village emergency care model, road optimization information and the location and number of newly constructed facility sites are determined. The disclosure improves the rationality of determining the location of a road section in need of transportation improvement and the number and location of emergency care service facilities, and improves the coverage rate and emergency care service efficiency of emergency care services under the input of limited cost in mountainous villages.

### BRIEF DESCRIPTION OF DRAWINGS

In order to more clearly illustrate the technical solutions and advantages in the embodiments or prior art of the disclosure, the accompanying drawings to be used in the description of the embodiments or prior art will be briefly introduced below, and it will be obvious that the accompanying drawings in the following description are only some of the embodiments of the disclosure, and that for those skilled in the art, other accompanying drawings can be obtained based on these accompanying drawings without putting in any creative labor.

FIG. 1 shows a flowchart of some embodiments of a method for selecting a location for an emergency care service facility in mountainous villages according to the disclosure;

FIG. 2 shows a schematic diagram of a process of solving a model for emergency care in mountainous villages by a modified simulated annealing algorithm according to the disclosure;

FIG. 3 shows a schematic diagram of the coding of the improved simulated annealing algorithm according to the disclosure;

FIG. 4 shows a schematic diagram of a single-point crossover method according to the disclosure;

FIG. 5 shows a schematic diagram of a neighborhood search generation process according to the disclosure.

### BEST MODE

In order to further elaborate the technical means and effects adopted by the disclosure to achieve the intended inventive purpose, the specific embodiments, structures, features and effects of the technical solutions based on the disclosure are described in detail as follows, taking into account the accompanying drawings and preferred embodiments. In the following description, different “one embodiment” or “another embodiment” does not necessarily refer to the same embodiment. In addition, particular features, structures or characteristics in one or more embodiments may be combined in any suitable form.

Unless otherwise defined, all technical and scientific terms used herein have the same meaning as commonly understood by those skilled in the art of the disclosure.

The disclosure provides a method for selecting a site for an emergency care service facility in mountainous villages, the method comprising the following steps:
obtaining mountainous village spatial data information;
determining a location corresponding to each of a plurality of alternative facility sites within a predetermined area based on the mountainous village spatial data information;
determining a mountainous emergency care efficiency set corresponding to each settlement in the predetermined area based on the mountainous village spatial data information;
constructing a mountainous village emergency care model based on locations corresponding to the plurality of alternative facility sites and the mountainous emergency care efficiency set corresponding to a settlement in the predetermined area; and
determining road optimization information and location and number of newly constructed facility sites based on the mountainous village spatial data information and the mountainous village emergency care model.

Each of the above steps is expanded in detail below.

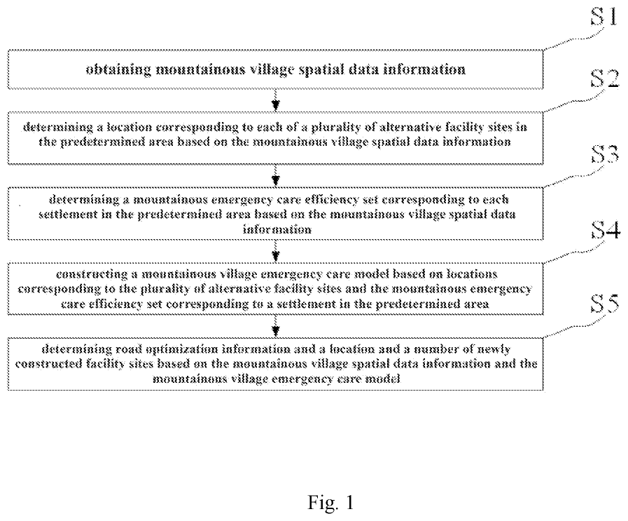

Referring to FIG. 1, a flow of some embodiments of a method for selecting a site of a mountain village emergency care service facility according to the disclosure is shown. The method for selecting a site for an emergency care service facility in mountainous villages comprises the following steps.

Step S1, obtaining mountainous village spatial data information.

In some embodiments, the mountainous village spatial data information may be obtained. Wherein, the above mountainous village spatial data information can be information corresponding to a geographic situation within a predetermined area. The mountainous village spatial data information comprises: settlement information corresponding to each settlement in the predetermined area, emergency care facility site information corresponding to an emergency care facility site, and road information corresponding to a road. The predetermined area may be a predetermined area where emergency care facility site additions and road improvements are required. The settlement information may include: a location of the settlement and a number of settlements within the settlement. The emergency care facility site information may be the location of the emergency care facility site. The road information may include: road condition and location. The emergency care facility site may be a location where the casualty or patient is acutely treated.

As an example, the step may include the following steps.

Step 1, obtaining historical spatial data information.

Where, the historical spatial data information is recorded and corresponds to a geographic situation in the predetermined area under a closest moment of a current moment.

For example, the current moment may be Jun. 27, 2022. The latest record concerning the geographic situation within the above predetermined area may be recorded on Jan. 1, 2022. Then, the record about the geographical situation within the above predetermined area recorded on Jan. 1, 2022 may be historical spatial data information.

Step 2, determining a location corresponding to each of a plurality of alternative facility sites in the predetermined area based on the mountainous village spatial data information.

In some embodiments, the location corresponding to the plurality of alternative facility sites in the above predetermined area may be determined based on the aforementioned mountainous village spatial data information.

Where, the alternative facility site is an emergency care facility site that may be newly constructed.

As an example, the step may include the following steps:

Step 1, dividing the predetermined area into a plurality of evaluation units.

Where, the evaluation unit may be an area in the predetermined area.

Step 2, determining a plurality of influencing factors and weights corresponding to the plurality of influencing factors.

Where, the influencing factor may be a factor affecting the siting of the newly constructed facility site. The newly constructed facility site may be an emergency care facility site that needs to be newly constructed.

For example, this step may include the following sub-steps.

In a first sub-step, determining the plurality of influencing factors.

Where, the number of influencing factors in the plurality of influencing factors may be seven.

For example, the plurality of influencing factors affecting the siting of the newly constructed facility site may include: three geographic factors, two transportation factors, one population distribution factor, and one hazard source distribution factor. The three geographic factors may be slope, elevation, and geologic hazard risk, respectively. Among them, the emergency care facility site should often be built on a slope less than 8° to facilitate rescue. The emergency care facility site should often be built in an area with an elevation of less than 500 meters above sea level. The emergency care facility site should be located away from areas prone to geologic hazards such as landslides and collapses. The two transportation factors can be the distance from a main road and the density of roads, respectively. The emergency care facility site should often be built in an area that are easily accessible and closer to main roads. The higher the road density, the more suitable it is for the construction of emergency care facility. The one population distribution factor can be the population density. The emergency care facility site should often be built in areas where the population is relatively concentrated. The one hazard distribution factor can be the distance from the source of the hazard. It is often inappropriate to construct an emergency care facility within the area of influence of the production and storage of hazardous materials, such as flammable and explosive materials.

In the second sub-step, determining the weights corresponding to the plurality of influencing factors.

For example, the weights corresponding to the plurality of influencing factors can be determined by hierarchical analysis.

Step 3, determining a plurality of indicators corresponding to each evaluation unit of the plurality of evaluation units based on the mountainous village spatial data information and the plurality of influencing factors.

Where, the indicator in the plurality of indicators may correspond one-to-one with the influencing factor in the plurality of influencing factors. The indicator may influence value corresponding to the factor.

For example, the plurality of indicators corresponding to the plurality of evaluation units may include: a slope of 7°, an elevation of 500 meters, a distance of 1,000 meters from a geologic hazard, a distance of 100 meters from a main road, 10 roads, 500 people, and a distance of 800 meters from a hazard source.

Step 4, determining an indicator impact score corresponding to each indicator of the plurality of indicators corresponding to each evaluation unit of the plurality of evaluation units based on the plurality of indicators corresponding to each evaluation unit of the plurality of evaluation units.

Where, the indicator impact score value corresponding to the indicator may be an evaluation value for the indicator. The higher the indicator impact score, the more suitable the evaluation unit corresponding to the indicator is for the construction of the emergency care facility. The value range of the indicator impact score can be [1, 10].

For example, through the fuzzy comprehensive evaluation method, the indicator impact score value corresponding to each indicator in the plurality indicators corresponding to each of the plurality of evaluation units.

Step 5, determining a total indicator impact score corresponding to each of the plurality of evaluation units based on the weights corresponding to the plurality of influencing factors and the indicator impact score corresponding to each indicator of the plurality of indicators corresponding to each of the evaluation units in the plurality of evaluation units.

For example, a formula for determining the total indicator impact score corresponding to each of the plurality of evaluation units corresponds to:

$${P}_{φ} = {\sum }_{σ = 1}^{n} {F}_{φ σ} \times {w}_{σ}$$

where P_{φ} is the total indicator impact score corresponding to φₜₕ evaluation unit, n is a number of influencing factors among the plurality of influencing factors, F_{φσ} is σₜₕ indicator corresponding to the φₜₕ evaluation unit, and w_{σ} is a weight corresponding to φₜₕ influencing factor.

Step 6, determining a location corresponding to each of the plurality of alternative facility sites in the predetermined area based on the total indicator impact score corresponding to the plurality of evaluation units.

For example, the plurality of evaluation units may be clustered into 4 categories based on the total indicator impact score corresponding to the evaluation unit. A mean value of the total indicator impact score corresponding to the evaluation unit in each of these 4 categories can be determined as the mean value of the impact corresponding to the category. The evaluation unit in the category that corresponds to the largest impact mean among these 4 categories can be identified as an alternative facility site.

For example, the evaluation units can be arranged in descending order according to the total indicator impact score corresponding to the evaluation unit to obtain the sequence of evaluation units. When there are four evaluation units in the sequence of evaluation units, the first evaluation unit can be taken as the alternative facility site, and the location of the first evaluation unit, that is, the location of the alternative facility site.

Step S3, determining a mountainous emergency care efficiency set corresponding to each settlement in the predetermined area based on the mountainous village spatial data information.

In some embodiments, the mountainous emergency care efficiency corresponding to each settlement within the above-described predetermined area may be determined based on the above-described mountainous village spatial data information.

As an example, the step may include the following steps.

Step 1, determining a time satisfaction of transferring a patient or an casualty in a settlement to each facility site to obtain a time satisfaction set corresponding to the settlement based on the mountainous village spatial data information.

Where, the facility site is an alternative facility site or an emergency care facility site.

For example, the present step may include the following sub-steps.

In the first sub-step, determining an access road set from the settlement to each facility site based on the mountainous village spatial data information.

Where the access road in the access road set may be a connected road. The access road from the settlement to the facility site may be a road leading from the settlement to the facility site.

In the second sub-step, determining a formular corresponding to a first time satisfaction set between the settlement and the facility site based on the access road set from the settlement to each facility site and a pre-obtained time spent cost of each road in the access road set as:

$$\text{?} = \text{?} {tr}_{ij}$$

$$\text{?} \text{indicates text missing or illegible when filed}$$

where G_{k,a,A} is the first time satisfaction of the patient or casualty in a kₜₕ settlement transferred to an Aₜₕ facility site in the predetermined area through an aₜₕ access road, the aₜₕ access road is an access road from the kₜₕ settlement to the Aₜₕ facility in the predetermined area, L_{k,a,A} is all the roads which make up the aₜₕ access road from the kₜₕ settlement to the Aₜₕ facility site in the predetermined area, trᵢⱼ is a time cost for the patient or casualty to pass through a road with road link (i,j) in L_{k,a,A,} and the first time satisfaction set between the kₜₕ settlement and the Aₜₕ facility site in the predetermined area includes: the first time satisfaction of the patient or casualty in the kₜₕ settlement who is transferred to the Aₜₕ facility site through each access road.

In the third sub-step, determining a smallest first time satisfaction in the first time satisfactions set from the settlement to each facility site as a reference time satisfaction from the settlement to the facility site.

In the fourth sub-step, combining reference time satisfactions from the settlement to each facility site into a reference time satisfaction set corresponding to the settlement.

Where, the reference time satisfaction in the reference time satisfaction set corresponding to the settlement may be a reference time satisfaction from the settlement to the facility site.

In the fifth sub-step, determining a largest reference time satisfaction in the reference time satisfaction set corresponding to the settlement as a control time satisfaction corresponding to the settlement.

In the sixth sub-step, determining a formula corresponding to the time satisfaction for a transfer of the patient or casualty from the settlement to each facility site based on the control time satisfaction corresponding to the settlement and the reference time satisfaction set from the settlement to each facility site as:

$${g}_{k , A} = \frac{{G}_{k} - {G}_{k , A}}{{G}_{k}}$$

where g_{k,A} is a time satisfaction for the transfer of the patient or casualty in the kₜₕ settlement to the Aₜₕ facility site in the predetermined area, Gₖ is a control time satisfaction corresponding to the kₜₕ settlement in the predetermined area, and G_{k,A} is a reference time satisfaction from the kₜₕ settlement to the Aₜₕ facility site in the predetermined area.

Step 2, determining road vulnerability coefficients of road sections through which the patient or casualty in the settlement is transferred to each facility site to obtain a road vulnerability coefficient set corresponding to the settlement based on the information of hidden roads included in the mountainous village spatial data information.

For example, a formula for determining the road vulnerability coefficient corresponding to the road section through which the patient or casualty in the settlement is transferred to each facility site is:

$${ρ}_{k , A} = \frac{{L}_{k , A}}{l}$$

where, ρ_{k,A} is a road vulnerability coefficient of the road section through which the patient or casualty from the kₜₕ settlement is transferred to the Aₜₕ facility site in the predetermined area, L_{k,A} is a number of potentially hazardous roads among all the roads from the kₜₕ settlement to the Aₜₕ facility site in the predetermined area, and l is a number of potentially hazardous roads among all the roads in the predetermined area.

Step 3, determining traffic satisfaction of the road sections through which the patient or casualty in the settlement are transferred to each facility site to obtain a traffic satisfaction set corresponding to the settlement based on traffic condition information included in the mountainous village spatial data information.

Where, the range of values of the traffic satisfaction may be [0, 1].

For example, the traffic satisfaction of the above settlement for transferring the patient or the casualty to the facility site can be determined by means of group decision-making based on the traffic condition information. The traffic satisfaction can be given by a plurality of decision makers separately, in which the maximum and minimum values in the traffic satisfaction can be removed, and the average value of the remaining traffic satisfaction in the traffic satisfaction can be taken as the traffic satisfaction between the settlement and the facility site.

Step 4, determining a mountainous emergency care efficiency set corresponding to the settlement based on the time satisfaction set, the road vulnerability coefficient set and the traffic satisfaction set corresponding to the settlement.

For example, the above formula for determining the mountainous emergency care efficiency set corresponding to the settlement is:

$${F}_{k , A} = \{ \begin{matrix}1 & {G}_{k , A} = 0 \\ ( 1 - {ρ}_{k , A} ) ( α {g}_{k , A} + β {λ}_{k , A} ) & {G}_{k , A} > 0\end{matrix}$$

where, F_{k,A} is an Aₜₕ mountainous emergency care efficiency in the mountainous emergency care efficiency set corresponding to the kₜₕ settlement in the predetermined area, α and β are weight coefficients, α∈[0,1], β∈[0.1], α+β=1, G_{k,A} is a reference time satisfaction from the kₜₕ settlement to the Aₜₕ facility site in the predetermined area, ρ_{k,A} is a road vulnerability coefficient of the road through which the patient or casualty of the kₜₕ settlement is transferred to the Aₜₕ facility site in the predetermined area, g_{k,A} is a time satisfaction of the patient or casualty in the kₜₕ settlement transferred to the Aₜₕ facility site in the predetermined area, and λ_{k,A} is a traffic satisfaction of the road section through which the patient or casualty in the kₜₕ settlement is transferred to the Aₜₕ facility site in the predetermined area.

Step S4, constructing a mountainous village emergency care model based on locations corresponding to the plurality of alternative facility sites and the mountainous emergency care efficiency set corresponding to a settlement in the predetermined area.

In some embodiments, the mountainous village emergency care model may be constructed based on the locations corresponding to the plurality of alternative facility sites and the mountainous emergency care efficiency set corresponding to the settlements in the aforementioned predetermined area.

As an example, the mountainous village emergency care model may be:

$$Number = Min {\sum }_{i ϵ N} {Z}_{i}$$

$$Efficiency = Max {\sum }_{k ϵ N} {d}_{k} {F}_{k}$$

$$Cost = Min \text{?} {c}_{ij} {X}_{ij} + {\sum }_{i ϵ N} {f}_{i} {Z}_{i}$$

$${Z}_{i} + {\sum }_{j ϵ N} {Y}_{ij}^{i} = 1 k ϵ N$$

$${\sum }_{k ϵ N , i \neq k} {\sum }_{j ϵ N} {Y}_{ji}^{k} {d}_{k} + {d}_{i} = {\sum }_{k ϵ N} {\sum }_{j ϵ N} {Y}_{ij}^{k} {d}_{k} i , k ϵ N , i \neq k , i ∉ F$$

$${Z}_{k} + {\sum }_{j ϵ N , i \neq k} {w}_{i}^{k} = 1 k ϵ N$$

$${\sum }_{( i , j ) ϵ L} {tr}_{ij} {Y}_{ij}^{k} \leq D k ϵ N$$

$${Y}_{ij}^{k} \leq {E}_{ij} ( i , j ) ϵ L , k ϵ N$$

$${W}_{i}^{k} \leq {Z}_{i} i , k ϵ N$$

$${Y}_{ij}^{i} + {Y}_{ji}^{j} \leq 1 ( i , j ) ϵ L$$

$$\text{?} \text{indicates text missing or illegible when filed}$$

Where, N is a number of settlements in the above predetermined area. ij,kϵ{1,2, . . . ,|N|}. F is a set of existing emergency care facility sites and proposed newly constructed facility sites. l is the set of existing roads and potential road links. dₖ is a number of patients and casualties in the kₜₕ settlement in the above predetermined area. dᵢ is a number of patients and casualties in the iₜₕ settlement in the above predetermined area. Fₖ is the mountainous emergency care efficiency between the kₜₕ settlement and the facility site. fᵢ is the cost of constructing facilities at i. cᵢⱼ is the cost of constructing or upgrading facilities at cᵢⱼ. trᵢⱼ is the cost of time spent by settlements on the road link (i,j). D is the critical emergency care time for the patient or casualty. Zᵢ is whether there is a facility site within the settlement i or not. When Zᵢ=1, there is a facility site within the settlement i. When Zᵢ=0, there is no facility site in settlement i. Zₖ is whether there is a facility site in settlement k. When Zₖ=1, there is a facility site within settlement k. When Zₖ=0, there is no facility site within settlement k. Eᵢⱼ is whether there is a new upgraded road or an existing road in link (i,j). When Eᵢⱼ=1, there is a new upgraded road or an existing road in link (i,j), and when Eij=0, there is no new upgraded road or an existing road in link (i,j). Xᵢⱼ is whether the road link (i,j) needs to be newly constructed or upgraded. When Xᵢⱼ=1, road link (i,j) needs to be newly constructed or upgrade. When Xᵢⱼ=0, the road link(i,j) does not need to be newly constructed or upgraded. Yᵢⱼ^{k} is whether the patient or the casualty in the iₜₕ settlement needs to go through the road link(i,j). when Yᵢⱼ^{k}=1, the patient or the casualty in the iₜₕ settlement needs to go through the road link(i,j). when Yᵢⱼ^{k}32 0, the patient or the casualty in the ith settlement does not need to go through the road link(i,j). Yⱼᵢ^{k} is whether the patient or casualty in the kₜₕ settelement needs to go through the road link (j,i). When Yⱼᵢ^{k}=1, the patient or casualty in the kₜₕ settlement needs to go through the road link (j,i), and when Yⱼᵢ^{k}=0, the patient or casualty in the kₜₕ settlement doesn't need to go through the road link (j,i). Yᵢⱼⁱ is whether the patient or casualty in the iₜₕ settlement needs to go through the road link (i,j). When Yᵢⱼⁱ=1, the patient or casualty in the iₜₕ settlement needs to go through the road link (i,j), and when Yᵢⱼⁱ=0, the patient or casualty in the iₜₕ settlement does not need to go through the road link (i,j). Wᵢ^{k} is whether or not the patient or casualty of the settlement k seeks the emergency care service at the facility site i. Number is to minimize the number of new emergency facilities. Efficiency is to maximize the total mountainous emergency care efficiency in the settlement. Cost is to minimize the cost of construction of emergency facilities as well as the cost of road upgrades. Zᵢ+Σ_{jϵN}Yᵢⱼⁱ=1 is the condition that settlement i is covered by at least one emergency facility. Σ_{kϵN,i‥k}Σ_{jϵN}Yⱼᵢ^{k}dₖ+dᵢ=Σ_{kϵN}Σ_{jϵN}Yᵢⱼ^{k}dₖ is the constraint that ensures the flow conservation. Zₖ+Σ_{jϵN,i≠k}Wᵢ^{k}=1 is the condition that there exists an emergency care facility site at the settlement k or that the patient or casualty at the settlement k needs to be transferred to other emergency care facility site. Σ_{(i,j)ϵL}trᵢₖYᵢⱼ^{k}≤D is the constraint that the time for settlement k to reach the nearest emergency care facility site is less than or equal to the set critical emergency time. Yᵢⱼ^{k}≤Eᵢⱼ is the constraint that the flow is available only on the already existing road links. Wᵢ^{k}≤Zᵢ is the constraint that the service can be accessed only in the area where the emergency care facility site exists. Yᵢⱼⁱ+Yⱼᵢ^{j}≤1 is a guarantee that it is a one-way link, i.e., the constraint that the patient or casualty in each settlement will not be transferred back and forth repeatedly. Number=MinΣ_{iϵN}Zᵢ, Efficiency=MaxΣ_{kϵN}dₖFₖ and Cost=MinΣ_{(i,j)ϵL}cᵢⱼXᵢⱼ+Σ_{iϵN}fᵢZᵢ can be the objective function.

Step S5, determining the road optimization information and the location and number of newly constructed facility sites based on the mountainous village spatial data information and the mountainous village emergency care model.

In some embodiments, the road optimization information and the location and number of the newly constructed facility sites may be determined based on the above mountainous village spatial data information and the above mountainous village emergency care model.

Where, the target facility site may be newly constructed emergency care facility site.

As an example, the road optimization information and the location and number of the newly constructed facility sites may be determined by the improved multi-objective simulated annealing algorithm based on the above-mentioned mountainous village spatial data information and the above-mentioned mountainous village emergency care model.

For example, the above mountainous village spatial data information can be substituted into the mountainous village emergency care model, and the road optimization information and the locations and numbers of the newly constructed facility sites can be determined by the improved multi-objective simulated annealing algorithm.

Among them, the road optimization information can be the road information that needs to be upgraded or newly constructed. The solution of the mountainous rural emergency care model may be the road optimization information and the location and number of newly constructed facility sites.

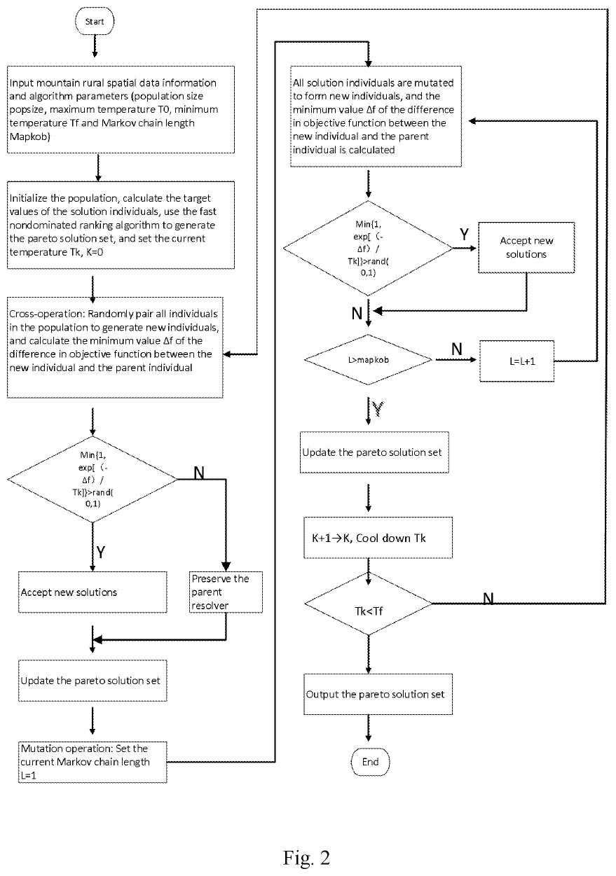

For example, the process of solving the mountainous village emergency care model by the improved simulated annealing algorithm may be shown in FIG. 2, which may include the following steps:

Step 1, determining the coding of the solution of the mountainous village emergency care model.

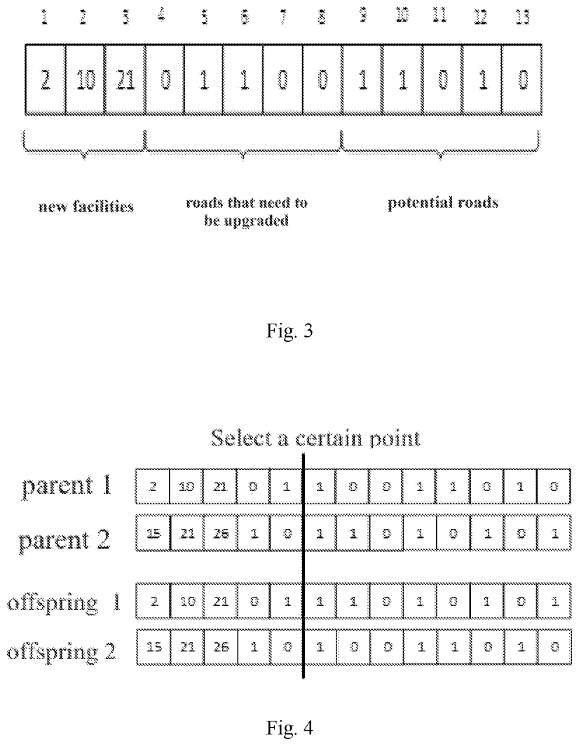

For example, the solution of the mountainous village emergency care model can be represented by an array, and the elements of this array can consist of the location numbers of the new emergency care service facilities and the numbers of 0 or 1. Where, 1 indicates upgrading of low-level or intermediate-level roads or new potential roads, and 0 indicates that there are no roads to upgrade or build. The new facility numbers, roads to be upgraded (low-level and high-level roads), and roads to be newly constructed are arranged in the front, center, and back of the array, respectively. As shown in FIG. 3, the above predetermined area can contain 3 new facilities, 5 roads that need to be upgraded, and 5 potential roads, then the coding of the solution of this mountainous village emergency care model can be represented as an array containing 13 elements. For example, the coding of the solution of this mountainous village emergency care model may be [2, 10, 21, 0, 1, 1, 0, 0, 0, 1, 1, 1, 0, 1, 0]. The first 3 elements of this array indicate the existence of 3 new facilities located on the settlements numbered 2, 10, and 21; the 4th through 8th elements indicate the upgrading of the 2nd and 3rd low-level or intermediate-level roads; and the 9th through 13th elements indicate the construction of the 1st, 2nd, and 4th roads.

Step 2, determining the initial solution.

In this algorithm, the generation of the initial population is randomized and the initial population is chosen as the basic solution set. Since an array is chosen here to represent the solution derived from the improved multi-objective simulated annealing algorithm, the array is randomly generated by the number of new facilities in the constraints. All the solutions inthe basic solution set are partitioned into sets of different categories using fast non-dominated sorting. And among the different sets, the top sorted set is selected as the initial Pareto optimal solution. Here, the purpose of fast non-dominated sorting is to divide all the individuals in the population into multiple sets based on their multidimensional objective function values by comparing them with each other according to the Pareto dominance relationship between them. There is no mutual dominance relationship among all individuals in the same set, while among different sets, individuals belonging to the top-ranked set have a Pareto dominance over those in the bottom-ranked set. The main computational procedure is that for each individual p contained in a given population P, the number of times it is dominated nₚ and the set of domination Sₚ are computed, where nₚ is the sum of the number of times each individual of pϵP is dominated by other individuals in the process of comparing it with other individuals. Sp is the set of domination of other individuals in the process of comparing it with other individuals for each individual of pϵP. set. Clearly, for all individuals of nₚ=0, the individual that is not dominated by any other individual is considered to be Pareto optimal solution. These individuals are deposited into the set F₁, which is the first-order solution; then all individuals in the set Sⱼ dominated by each individual j in F₁ are examined, and the number of dominated times nₖ of all individuals in the set Sⱼ is subtracted from 1 to denote the number of dominations of individual k by individuals of the remaining portion of the population after taking out the set F₁. For the remaining population, all individuals with the number of dominated times nₚ=0 then form the second-order solution and are deposited in the set F₂. Repeat the above steps until all individuals are deposited in the set of the corresponding rank. Use F1 as the first rank set of non-dominated solutions and assign each individual in this set the same domination order number rank=1, all individuals in F2 are assigned rank=2. and so on until all individuals in the population are ranked.

Step 3, performing reorganize operation.

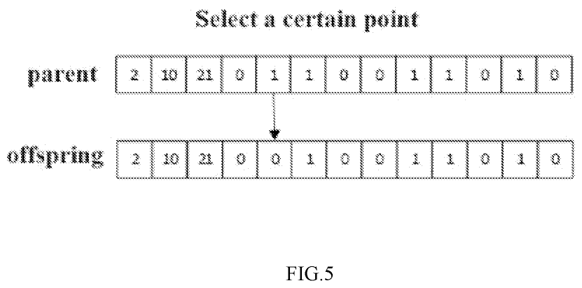

The purpose of the reorganize operation is to regenerate a new offspring from the parent, which is equivalent to the crossover operation inside the genetic algorithm, and a single point crossover method can be used to generate a new solution. Here, the single point crossover method is used to generate a new solution, and the single point crossover method can be shown in FIG. 4. Select a certain point in the array, and then divide this into left and right parts, and reorganize the right part of the two arrays by exchanging element sequences with each other. If duplicate facility sites appear in the reorganized solution individual, delete the duplicate facility sites first, and then supplement a newly constructed facility site in a random way.

Step 4, generating neighborhood solution.

Generating neighborhood solution is equivalent to the mutation operation in genetic algorithm, in which randomly pick a position in the parent solution array and change the value of the position so as to generate the child solution array, and the neighborhood search generation process can be shown in FIG. 5.

Step 5, performing Metropolis acceptance criterion.

After obtaining a new neighborhood solution, the next step is to determine whether this solution will be accepted in the next iteration process. The minimum value of the difference between the objective function value corresponding to the child generation solution and the objective function value corresponding to the parent generation solution is calculated to determine whether the new solution will be accepted or not. For example, two parent solutions X=(x₁, x₂, . . . , xₙ) and Y=(y₁, y₂, . . . , yₙ) generate two child solutions X¹=(x₁¹, x₂², . . . ,, xₙ¹) and Y¹=(y₁¹, y₂², . . . , yₙ¹). Then the objective function 1, objective function 2 and objective function 3 values corresponding to the parent generation solution X are denoted by fₙ(X), fₑ(X) and f_{c}(X), respectively; the objective function 1, objective function 2 and objective function 3 values corresponding to the parent generation solution Y are denoted by fₙ(Y), fₑ(Y) and f_{c}(Y), respectively. The objective function 1, objective function 2 and objective function 3 values corresponding to the child generation solution X¹ are denoted by fₙ(X¹), fₑ(X¹) and f_{c}(X¹) , respectively; and the objective function 1, objective function 2 and objective function 3 values corresponding to the child generation solution Y¹ are denoted by fₙ(Y¹), fₑ(Y¹) and f_{c}(Y¹), respectively. Then, Δf₁ and Δf₂ can be calculated according to the following two formulas, respectively, and the Metropolis criterion can be used to determine whether the offspring solution is accepted or not.

Δf₁=min{fₙ(X¹)−fₙ(X),fₙ(X¹)−fₙ(Y),fₑ(X¹)−fₑ(X),fₑ(X¹)−fₑ(Y),f_{c}(X¹)−f_{c}(X),f_{c}(X¹)−f_{c}(Y)}

Δf₂=min{fₙ(Y¹)−fₙ(X),fₙ(Y¹)−fₙ(Y),fₑ(Y¹)−fₑ(X),fₑ(Y¹)−fₑ(Y),f_{c}(Y¹)−f_{c}(X),f_{c}(Y¹)−f_{c}(Y)}

Step 6, performing the temperature reduction operation.

For example, use Tₙ to denote the nₜₕ temperature iteration, then the (n+1)ₜₕ temperature iteration can be expressed as Tₙ₊₁=α×Tₙ, wherein α is the rate of temperature reduction and 0<α<1. Increasing the value of α will slow down the rate of temperature reduction.

The method for selecting the location of emergency care service facility in mountainous village of the disclosure improves the rationality of the location of a road section in need of transportation improvement and the location and number of emergency care service facilities, and improves the coverage rate and emergency care service efficiency of the emergency care service under the input of limited cost in the mountainous village. First, a spatial data information of the mountainous village is obtained, wherein the afore spatial data information of the mountainous village is information corresponding to a geographic situation within a predetermined area, and the afore spatial data information of the mountainous village comprises: information of a settlement corresponding to each settlement within the afore predetermined area, information of an emergency care facility site corresponding to the emergency care facility site, and information of a road corresponding to the road. In fact, for selecting the location of the emergency care facility site, it is often necessary to obtain information about the area in which the emergency care facility site needs to be added, which can facilitate comparison of the geographic conditions of different areas in the predetermined area, and can improve the accuracy of the subsequent selection of the location of the emergency care facility site. Next, based on the afore spatial data information of the mountainous village, a location corresponding to a plurality of alternative facility sites within the above predetermined area is determined. A plurality of factors are considered in a comprehensive manner, which improves the accuracy of the preliminary determination of the locations corresponding to the alternative facility sites. Then, based on the above mountainous village spatial data information, a mountainous emergency care efficiency set corresponding to each settlement within the above predetermined area is determined. Afterwards, mountainous villages emergency care model is constructed based on the locations corresponding to the plurality of alternative facility sites and the mountainous emergency care efficiency set corresponding to the settlements within the above-described predetermined area. Finally, based on the above mountainous village spatial data information and the above mountainous village emergency care model, road optimization information and the location and number of newly constructed facility sites are determined. The disclosure improves the rationality of determining the location of a road section in need of transportation improvement and the number and location of emergency care service facilities, and improves the coverage rate and emergency care service efficiency of emergency care services under the input of limited cost in mountainous villages.

The above-described embodiments are only used to illustrate the technical solutions of the present application, not to limit them; although the present application has been described in detail with reference to the foregoing embodiments, those skilled in the art should understand that it is still possible to modify the technical solutions documented in the foregoing embodiments or to replace some of the technical features therein by equivalent ones; and such modifications or replacements do not detach the essence of the corresponding technical solutions from the spirit and scope of the technical solutions of the embodiments of the present application, and shall be included in the scope of protection of the present application.

1. A method of siting emergency medical service facilities in mountain villages, comprising:
obtaining mountainous village spatial data information, wherein the mountainous village spatial data information is information corresponding to a geographic situation within a predetermined area, and the mountainous village spatial data information comprises: settlement information corresponding to each settlement in the predetermined area, emergency care facility site information corresponding to an emergency care facility site, and road information corresponding to a road;
determining a location corresponding to each of a plurality of alternative facility sites in the predetermined area based on the mountainous village spatial data information;
determining a mountainous emergency care efficiency set corresponding to each settlement in the predetermined area based on the mountainous village spatial data information;
constructing a mountainous village emergency care model based on locations corresponding to the plurality of alternative facility sites and the mountainous emergency care efficiency set corresponding to a settlement in the predetermined area; and
determining road optimization information and a location and a number of newly constructed facility sites based on the mountainous village spatial data information and the mountainous village emergency care model.

2. The method of siting emergency medical service facilities in mountain villages of claim 1, wherein the obtaining mountainous village spatial data information, comprising:
obtaining historical spatial data information, wherein the historical spatial data information is recorded and corresponds to a geographic situation in the predetermined area under a closest moment of a current moment;
obtaining, by means of a remote sensing device, a remote sensing image in the predetermined area under the current moment; and
updating the historical spatial data information based on the remote sensing image to obtain the mountainous village spatial data information.

3. The method of siting emergency medical service facilities in mountain villages of claim 1, wherein the determining the location corresponding to each of the plurality of alternative facility sites in the predetermined area based on the mountainous village spatial data information, comprising:
dividing the predetermined area into a plurality of evaluation units;
determining a plurality of influencing factors and weights corresponding to the plurality of influencing factors;
determining a plurality of indicators corresponding to each evaluation unit of the plurality of evaluation units based on the mountainous village spatial data information and the plurality of influencing factors;
determining an indicator impact score corresponding to each indicator of the plurality of indicators corresponding to each evaluation unit of the plurality of evaluation units based on the plurality of indicators corresponding to each evaluation unit of the plurality of evaluation units;
determining a total indicator impact score corresponding to each of the plurality of evaluation units based on the weights corresponding to the plurality of influencing factors and the indicator impact score corresponding to each indicator of the plurality of indicators corresponding to each of the evaluation units in the plurality of evaluation units; and
determining a location corresponding to each of the plurality of alternative facility sites in the predetermined area based on the total indicator impact score corresponding to the plurality of evaluation units.

4. The method of siting emergency medical service facilities in mountain villages of claim 3, wherein a formula for determining the total indicator impact score corresponding to each of the plurality of evaluation units corresponds to:
$${P}_{φ} = {\sum }_{σ = 1}^{n} {F}_{φσ} \times {w}_{σ}$$
where P_{φ} is the total indicator impact score corresponding to φₜₕ evaluation unit, n is a number of influencing factors among the plurality of influencing factors, F_{φσ} is σₜₕ indicator corresponding to the φₜₕ evaluation unit, and w_{σ} is a weight corresponding to σₜₕ influencing factor.

5. The method of siting emergency medical service facilities in mountain villages of claim 1, wherein the determining the mountainous emergency care efficiency set corresponding to each settlement in the predetermined area based on the mountainous village spatial data information, comprising:
determining a time satisfaction of transferring a patient or an casualty in a settlement to each facility site to obtain a time satisfaction set corresponding to the settlement based on the mountainous village spatial data information, wherein the facility site is an alternative facility site or an emergency care facility site;
determining road vulnerability coefficients of road sections through which the patient or casualty in the settlement is transferred to each facility site to obtain a road vulnerability coefficient set corresponding to the settlement based on the information of hidden roads included in the mountainous village spatial data information;
determining traffic satisfaction of the road sections through which the patient or casualty in the settlement are transferred to each facility site to obtain a traffic satisfaction set corresponding to the settlement based on traffic condition information included in the mountainous village spatial data information; and
determining a mountainous emergency care efficiency set corresponding to the settlement based on the time satisfaction set, the road vulnerability coefficient set and the traffic satisfaction set corresponding to the settlement.

6. The method of siting emergency medical service facilities in mountain villages of claim 5, wherein the determining the time satisfaction for transferring a patient or casualty in the settlement to each facility site based on the mountainous village spatial data information, comprising:
determining an access road set from the settlement to each facility site based on the mountainous village spatial data information;
determining a formular corresponding to a first time satisfaction set between the settlement and the facility site based on the access road set from the settlement to each facility site and a pre-obtained time spent cost of each road in the access road set as:
$$\text{?} = \text{?} {tr}_{ij}$$
$$\text{?} \text{indicates text missing or illegible when filed}$$
where G_{k,a,A} is the first time satisfaction of the patient or casualty in a kₜₕ settlement transferred to an Aₜₕ facility site in the predetermined area through an aₜₕ access road, the aₜₕ access road is an access road from the kₜₕ settlement to the Aₜₕ facility in the predetermined area, L_{k,a,A} is all the roads which make up the aₜₕ access road from the kₜₕ settlement to the Aₜₕ facility site in the predetermined area, trᵢⱼ is a time cost for the patient or casualty to pass through a road with road link (i,j) in L_{k,a,A,} and the first time satisfaction set between the kₜₕ settlement and the Aₜₕ facility site in the predetermined area includes: the first time satisfaction of the patient or casualty in the kₜₕ settlement who is transferred to the Aₜₕ facility site through each access road;
determining a smallest first time satisfaction in the first time satisfactions set from the settlement to each facility site as a reference time satisfaction from the settlement to the facility site;
combining reference time satisfactions from the settlement to each facility site into a reference time satisfaction set corresponding to the settlement;
determining a largest reference time satisfaction in the reference time satisfaction set corresponding to the settlement as a control time satisfaction corresponding to the settlement; and
determining a formula corresponding to the time satisfaction for a transfer of the patient or casualty from the settlement to each facility site based on the control time satisfaction corresponding to the settlement and the reference time satisfaction set from the settlement to each facility site as:
$${g}_{k , A} = \frac{{G}_{k} - {G}_{k , A}}{{G}_{k}}$$
where g_{k,A} is a time satisfaction for the transfer of the patient or casualty in the kₜₕ settlement to the Aₜₕ facility site in the predetermined area, Gₖ is a control time satisfaction corresponding to the kₜₕ settlement in the predetermined area, and G_{k,A} is a reference time satisfaction from the kₜₕ settlement to the Aₜₕ facility site in the predetermined area.

7. The method of siting emergency medical service facilities in mountain villages of claim 5, wherein a formula for determining the road vulnerability coefficient corresponding to the road section through which the patient or casualty in the settlement is transferred to each facility site is:
$${ρ}_{k , A} = \frac{{L}_{k , A}}{l}$$
where ρ_{k,A} is a road vulnerability coefficient of the road section through which the patient or casualty from the kₜₕ settlement is transferred to the Aₜₕ facility site in the predetermined area, L_{k,A} is a number of potentially hazardous roads among all the roads from the kₜₕ settlement to Aₜₕ facility site in the predetermined area, and l is a number of potentially hazardous roads among all the roads in the predetermined area.

8. The method of siting emergency medical service facilities in mountain villages of claim 5, wherein a formula for determining the mountainous emergency care efficiency set corresponding to the settlement is:
$${F}_{k , A} = \{ \begin{matrix}1 & {G}_{k , A} = 0 \\ ( 1 - {ρ}_{k , A} ) ( α {g}_{k , A} + β {λ}_{k , A} ) & {G}_{k , A} > 0\end{matrix}$$
where F_{k,A} is an Aₜₕ mountainous emergency care efficiency in the mountainous emergency care efficiency set corresponding to the kₜₕ settlement in the predetermined area, α and β are weight coefficients, α∈[0,1], β∈[0,1], α+β=1, G_{k,A} is a reference time satisfaction from the kₜₕ settlement to the Aₜₕ facility site in the predetermined area, ρ_{k,A} is a road vulnerability coefficient of the road through which the patient or casualty of the kₜₕ settlement is transferred to the Aₜₕ facility site in the predetermined area, g_{k,A} is a time satisfaction of the patient or casualty in the kₜₕ settlement transferred to the Ath facility site in the predetermined area, and λ_{k,A} is a traffic satisfaction of the road section through which the patient or casualty in the kₜₕ settlement is transferred to the Aₜₕ facility site in the predetermined area.

9. The method of siting emergency medical service facilities in mountain villages of claim 1, wherein the determining road optimization information and the location and the number of newly constructed facility sites based on the mountainous village spatial data information and the mountainous village emergency care model comprises:
determining the road optimization information and the location and number of newly constructed facility sites based on the mountainous village spatial data information and the mountainous village emergency care model, by means of an improved multi-objective simulated annealing algorithm.
